(19) United States
(12) Patent Application Publication
(10) Pub. No.: US 2005/0222117 A1
(43) Pub. Date: Oct. 6, 2005

(54) Chlorophyll and its derivatives for cancer photodynamic therapy

(75) Inventors: Yih-Chih Hsu, Taipei City (TW); Wen-Tyng Li, Taipei City (TW); Jeffrey Lin, Jacksonville, FL (US); Bor-Iuan Jan, Pingtung City (TW); Tzu-I Wu, Shenkong Shiang (TW); Jih-Shen Wen, Taipei City (TW); Chi-Ming Yang, Taipei City (TW)

Correspondence Address: BACON & THOMAS, PLLC, 625 SLATERS LANE, FOURTH FLOOR, ALEXANDRIA, VA, 22314, US

(73) Assignee: Sing Land Biotech International Co., Ltd., Taipei (TW)

(21) Appl. No.: 11/007,325

(22) Filed: Dec. 9, 2004

(30) Foreign Application Priority Data
Mar. 31, 2004 (TW) 093108918
Mar. 31, 2004 (TW) 093108921

(51) Int. Cl. A61K 31/409; A61K 31/555

(52) U.S. Cl. 514/185; 514/410

(57) ABSTRACT

The present invention relates to a method for treating liver cancer or oral cancer through photodynamic therapy, comprising administrating a subject in need thereof an effective amount of formula I, formula □, formula III or formula IV, which could release reactive oxygen species to inhibit the growth of cancer cells, which comprise with formulas as described in the specification, wherein R1 is an alkyl or an aldehyde with carbon atoms no more than 2; the cancer is liver cancer or oral cancer.

### BACKGROUND OF THE INVENTION

1. Field of the Invention

The present invention relates to a method and a pharmaceutical composition, which could release reactive oxygen species to inhibit the growth of cancer cells.

2. Description of Related Art

Photodynamic therapy is a new therapeutic method. When a photosensitive material is excited with high radiation energy, it will drop from an excited state to its ground state accompanying a series of chemical reactions and release of energy which activates oxygen molecules in cells into reactive oxygen species. In such case, the integrity and permeability of cell membrane and the function of membrane proteins will be destroyed. Also, the enzyme function within inner and outer membranes of the mitochondria will be inhibited. The above process will result in lower amount of ATP production in a cell, while destroying microsomes and normal enzyme functions within the plasma membrane, thereby achieving the result of cell death.

Photodynamic effects can be applied in disease therapy with the features of specific targeting on abnormal tissue which intakes and retains more photosensitive agent than normal tissue. The targeted tissue will behave strongly in photodynamic effects after irradiation, and thus the abnormal tissue can be destroyed. In fact, malignant tumor, some photosensitive agent inside them. The abnormal tissues within the reaching range of the laser optical fiber may be treated with photodynamic therapy.

During photodynamic therapy, single oxygen and free radicals produced from the process of light-energy conversion can directly cause damage to the cells of disordered tissue. Moreover, this process can also induce partial microcirculation obstruction by causing lesion of capillary vessel endothelium and vessel embolism, which further results in necrosis of disordered tissues. In addition, recent research indicated activation of immune system is one of the important mechanism to cause cell death during photodynamic therapy.

The reason why cancer cells retain higher concentration of photosensitizing agent than normal cells may be due to the structural difference between neoplasm and normal tissue. Neoplasm has leaky capillary network, fast cell proliferation rate, lower pH, and poor lymphoid fluid circulation. Some cancer cell expresses higher level of LDL receptor on the cell membrane. Photosensitizing agent is often chemically modified to enhance its selectivity to cancer cells. For example, the membrane permeability of photosensitizing agent can be improved by increasing its hydrophobicity. Photosensitizing agent can easily target cancer cells which express high amount of LDL receptor by applying photosensitizer conjugated with LDL. The selectivity to cancer cells can be enhanced by using photosensitizer conjugated with specific cancer cell markers. The photosensitizing agent suitable for photodynamic therapy should have the receiving light irradiation, lower amount of retention within normal tissue and specific target to neoplasm.

Most of photosensitizing agents on development are chemically synthesized and has high selectivity between neoplasm and normal tissue. However, their cost is expensive. Drug delivery carriers such as liposome must be formulated due to hydrophobicity of most photosensitizing agents. Early generation of photosensitizing agents have the adverse effects during photodynamic therapy including photosensitivity, heat reaction, phototoxicity, and skin pigmentation. Though photodynamic therapy has been applied in clinical cancer therapy, prior art had not specified the mechanism and efficacy of treatment to specific target cancer cells. Current effort on developing photosensitizing agents is mostly focusing on their modification. In addition, light source with lower cost, photosensitizing agents derived from natural products and cheaper photosensitizing agents are also in active pursuit. It is important to provide more evidence regarding the efficacy of photosensitizing agent to specific cancer.

### SUMMARY OF THE INVENTION

The object of the present invention is to provide a method for treating cancer through photodynamic therapy. Upon absorption of red light, the compound should be able to release reactive oxygen species and can be used to treat liver or oral cancer through photodynamic therapy.

The other object of the present invention is to provide a and is suitable for application in drug delivery as a water-soluble form for oral cancer treatment.

Another object of the present invention is to provide a compound along with its pharmaceutical composition that is able to selectively accumulate itself within cancer cells, therefore achieving the goals as a cancer diagnostic tool and a tool for visualizing the site of cancer.

To achieve the above objects, the present invention provides a method for treating liver cancer through photodynamic therapy, comprising of administrating a subject in need thereof with an effective amount of formula I: 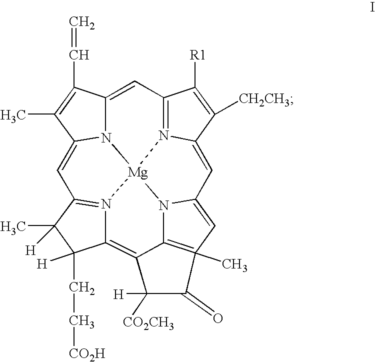
Wherein, R1 is an alkyl or an aldehyde with carbon atoms no more than 2; the disease treated is liver cancer.

Another method for treating cancer through photodynamic therapy, comprising administrating a subject in need thereof with an effective amount of formula □, formula III and formula IV: 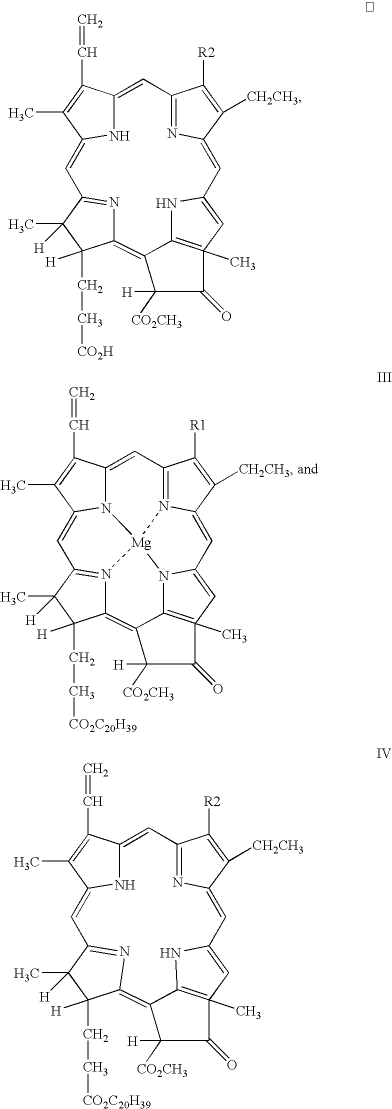
wherein, R1 and R2 is independently an alkyl or an aldehyde with carbon atoms no more than 2; said cancer applied is oral or liver cancer.

Also, the present invention provides a compound for cancer photodynamic therapy comprising of at least one structure from formula □, formula III and formula IV: 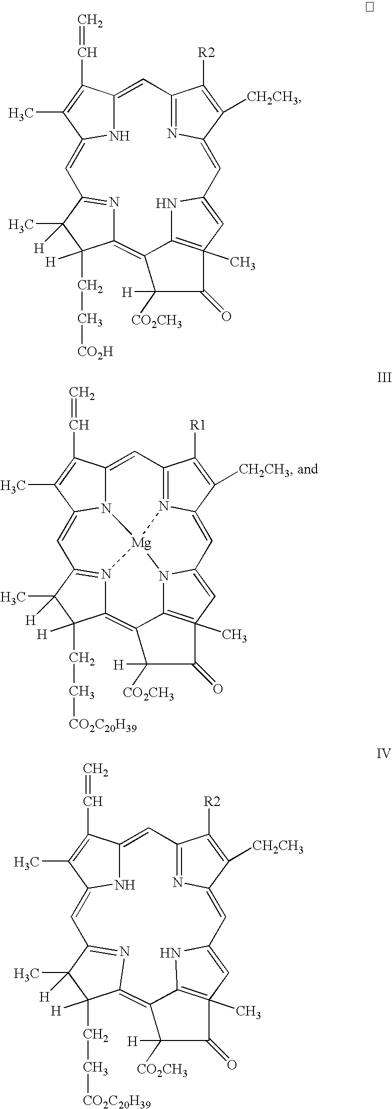
wherein, R1 and R2 are either alkyl or aldehyde with no more than 2 carbon atoms; and the cancer treated illustrated above is either oral or liver cancer.

The present invention further relates to a pharmaceutical composition for cancer photodynamic therapy, which comprises of at least one compound from formula I, □ formula □, formula III and formula IV: 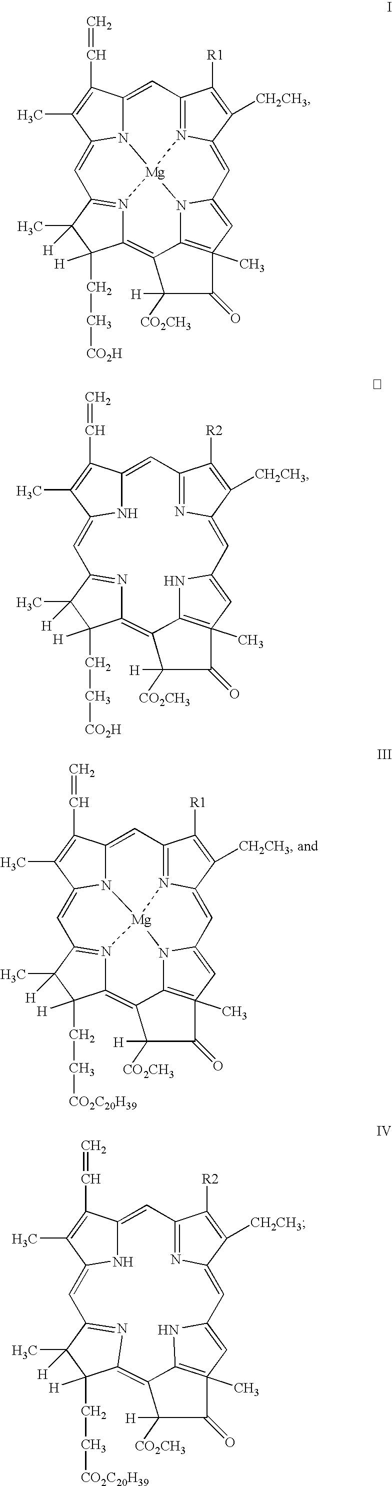
wherein, R1 and R2 are either alkyl or aldehyde with carbon atoms no more than 2; and the cancer illustrated above can be either oral cancer or liver cancer.

In the present invention, R1 is preferably CH₃ or CHO; and the effective wavelength for the compound is not limited; preferably, the effective spectral region ranges from 600 to 800 nm. The compounds disclosed in the present invention are able to accumulate in cancer cells, and can be excited with appropriate wavelength. The compound is suitable for use in diagnostic and localization of cancer cells.

The excited light source suitable for compounds of the present invention can be red-light source which is better in penetrating cells or tissues compared to other light sources, and the advantage is beneficial in photodynamic therapy.

Other objects, advantages, and novel features of the invention will become more significant from the following detailed description when taken in conjunction with the accompanied drawings.

### BRIEF DESCRIPTION OF THE DRAWINGS

FIG. 1 is a bar chart cytotxicity of cancer and normal cells after receiving the treatment with compounds of the present invention and light irradiation;

FIG. 2 is a bar chart of the ratio of cancer and normal cell survival after receiving the treatment with compounds of the present invention and light irradiation;

FIG. 3 showed fluorescence images of cancer cells after receiving the treatment with compounds of the present invention with (panel A) or without (panel B) light irradiation. The nuclei (lane 1) and mitochondria (lane 2) have been photo-destructed after photodynamic treatment.

### DETAILED DESCRIPTION OF THE PREFERRED EMBODIMENT

The compound of present invention for cancer photodynamic therapy discloses several features in the following examples: the structures of the present invention are novel photosensitive structures, and the efficiency in inhibiting the growth of human liver hepatoma cells and oral carcinoma cells are also approved in these examples described below. In addition, the examples also showed analysis data of nuclei and cell membrane destruction after receiving photodynamic treatment of present invention compounds, in which established the mechanism of photodynamic therapy.

### EXAMPLE 1

The compounds of the present invention were prepared according to the modified method of Omata T. and Murata N.'s “Preparation of chromatography with DEAE CL-6B and sepharose CL-6B” (Plant Cell Physiol. (1983) 24:1093-1100).

The 8 embodiments of the present invention with formulas I a, I b, II a, II b, III a, III b, IVa and IVb were prepared:

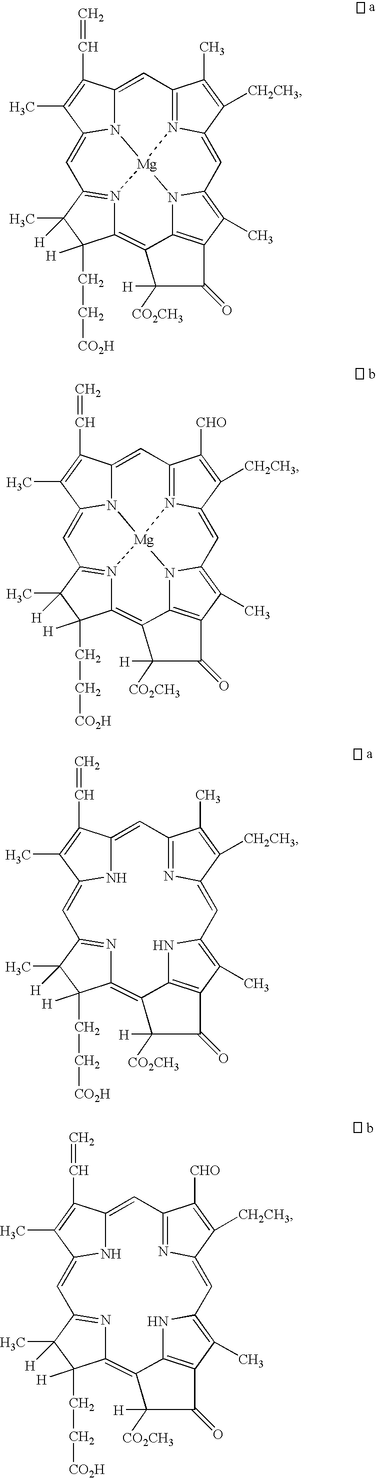

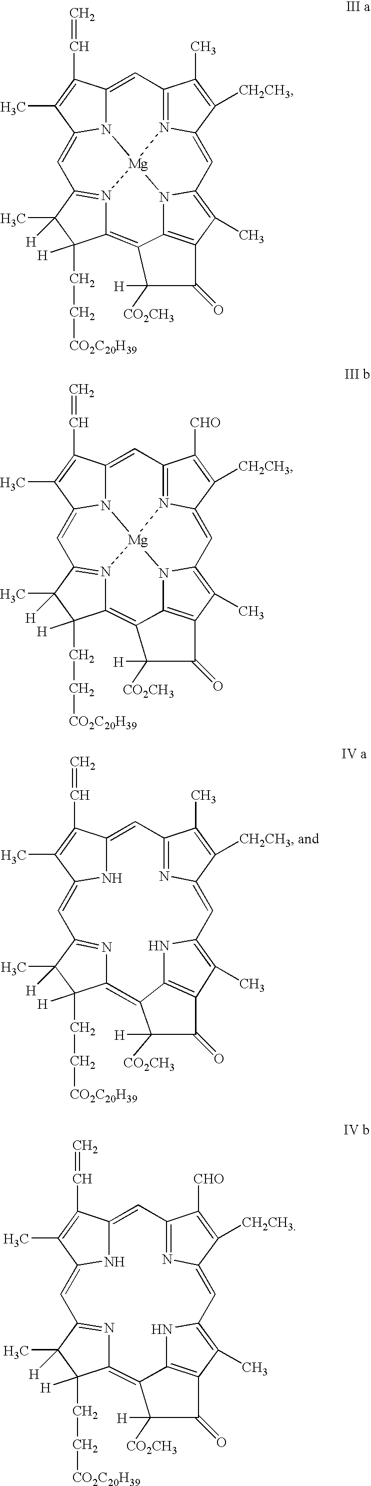

### EXAMPLE 2

To validate the maximum absorption wavelength of the present invention, those 8 compounds obtained from example 1 were diluted with double distilled water to concentrations of 0.25 mg/ml and 0.5 mg/ml for further examination. Each diluted sample was placed in a quartz cuvette and scanned from 300 to 1000 nm for maximum optical absorption wavelength by a spectrophotometer. The data is shown as table 1.

TABLE 1

| compound | λmax(nm) |
|---|---|
| □a | 669 |
| □b | 662 |
| □a | 668 |
| □b | 666 |
| IIIa | 676 |
| IIIb | 698 |
| IVa | 663 |
| IVb | 698 |

According to the data of table 1, it is clearly established that the preferable spectral regions of the compounds of the present invention is 600-700 nm.

### EXAMPLE 3

Ratios of cells which took up 8 compounds obtained from example 1 were estimated by flow cytometry. The detailed procedures of the estimation method are described below: Cells including human foreskin fibroblast, human hepatoma cell line—HepG2/C3A, and oral carcinoma cell line—SCC-4 were seeded at density of 2×10⁵ cells per well into a 6-well cell culture plate. Human foreskin fibroblast cells were cultured in MEM medium (Gibco 61100-061) supplement with 10% fetal bovine serum (FBS). HepG2/C3A cells were cultured in DMEM medium (Gibco 12100-046) containing 10% FBS. SCC-4 cells were cultured in DMEM/F12 medium (Gibco 12100-024) supplied with 10% FBS. All cells were incubated overnight in 100% humidified environment of 37° C., 5% CO₂ allowing cell attachment.

The cultured cells were washed twice with PBS buffer. Serum free culture medium was added, and then 1.25 μg/ml of 8 compounds was treated for 150 minutes in the dark.

The cells were trypsinized, washed with FACS buffer, and 2% of formaldehyde (Sigma) was added to fix cells. After cell fixation, solution was washed off with FACS buffer, the cells were suspended in 2 ml FACS buffer. The experiment was performed by flow cytometry within 24 hours. The excitation wavelength was 488 nm. Cells without treatment with compounds in the present invention were taken as control group. The ratios of cells which took up the compounds were summarized in Table 2a and Table 2b.

TABLE 2a

| compound | human foreskin fibroblast | SCC-4 | HepG2/C3A |
|---|---|---|---|
| □a | 48.8% | 69.1% | 73.5% |
| □b | 65.7% | 79.3% | 78.0% |
| IIIa | 47.5% | 81.5% | 54.3% |
| IIIb | 62.3% | 86.1% | 55.7% |
| IVa | 86.8% | 95.3% | 85.4% |
| IVb | 70.8% | 94.1% | 80.5% |

TABLE 2b

| compound | human foreskin fibroblast | HepG2/C3A |
|---|---|---|
| Ia | 39.9% | 42.7% |
| Ib | 44.3% | 64.7% |

According to Table 2a, the ratio of cancer cells uptake all 6 compounds was higher than that of normal cells. Among them, the ratio of oral carcinoma cell line—SCC-4 absorbed IV a and IV b compounds was the highest, and the ratio of hepatoma cell line—HepG2/C3A absorbed IV a and IV b compounds was higher than that of human foreskin fibroblasts. The efficiency of compounds III a and III b passing through cell membrane of liver cancer cell was equal to that of fibroblast cells. Table 2b indicates the ratio of HepG2/C3A uptake compound Ia and Ib was higher then that of normal cells.

### EXAMPLE 4

MTT assay was used to estimate the effect on cell activity with photosensitive drugs. 10 thousands of cells including human foreskin fibroblasts, HepG2/C3A cells, and SCC-4 cells were placed into each well of a 96-well cell culture plate. Human foreskin fibroblasts were cultured in MEM medium (Gibco 61100-061) supplied with 10% FBS. The liver hepatoma cell line—HepG2/C3A were cultured in DMEM medium (Gibco 12100-046) supplement with 10% FBS. The oral carcinoma cell line—SCC-4 were cultured in DMEM/F12 medium (Gibco 12100-024) with 10% FBS. All cells were incubated overnight at 100% humidity, 37° C., 5% CO₂ allowing cell attachment.

The cultured cells were washed twice with PBS. Serum free medium containing 1.25 μg/ml of 8 compounds was added to treat cells for 150 minutes in the dark. After treatment, the drug solution was washed off and the cells were rinsed twice with PBS. Then the medium was then replaced with serum free medium, and the cells were irradiated with 680nm red-light for 20 minutes (accumulated total energy 16 J/cm²) or 30 minutes (accumulated total energy 24 J/cm²).

After irradiation, serum-free medium was then replaced with culture medium with 10% FBS and continued culturing. The MTT assay was performed 2 days after light irradiation. First, the supernatant of cell culture was discarded. Then the 96-well plate was washed twice with PBS. Each well was added with 0.1 ml of MTT solution (0.5 mg/ml) (3-[4,5-dimethylthiazol-2-yl]-2,5-diphenyl tetrazolium bromide, Sigma), then incubated in 37° C. for 3 hours. Formazan crystals of purple color would form at the bottom of each well. The supernatant of MTT was discarded, and 0.1 ml of DMSO was then added into each well to dissolve formazan crystals. The crystals were dissolved completely after 5-10 minutes, the absorbance was then determined at wavelength of 560 nm with an ELISA the mean values were calculated. Cell survival ratio=absorbance _{light irradiation with compound treatment}/absorbance _{light irradiation without compound treatment}.

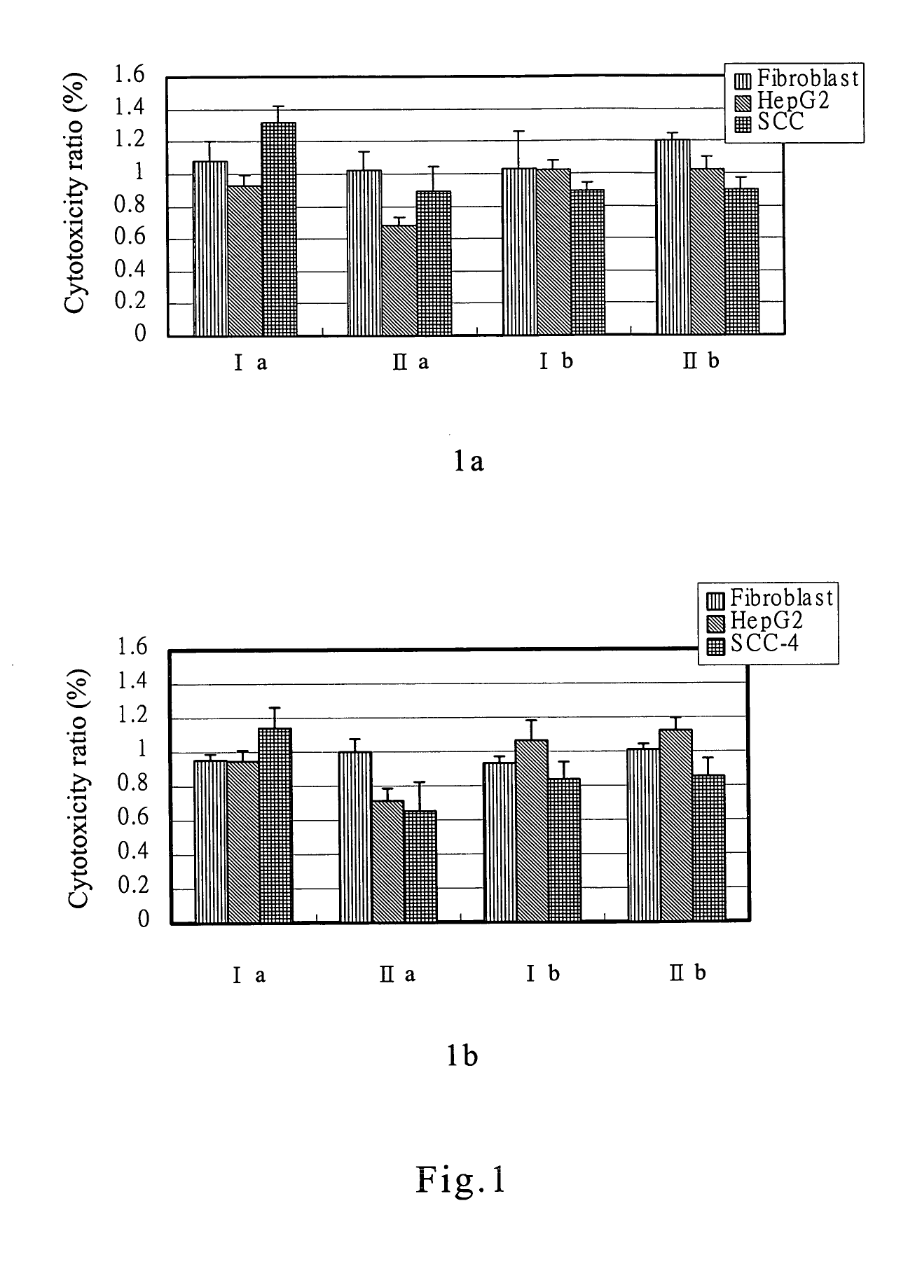

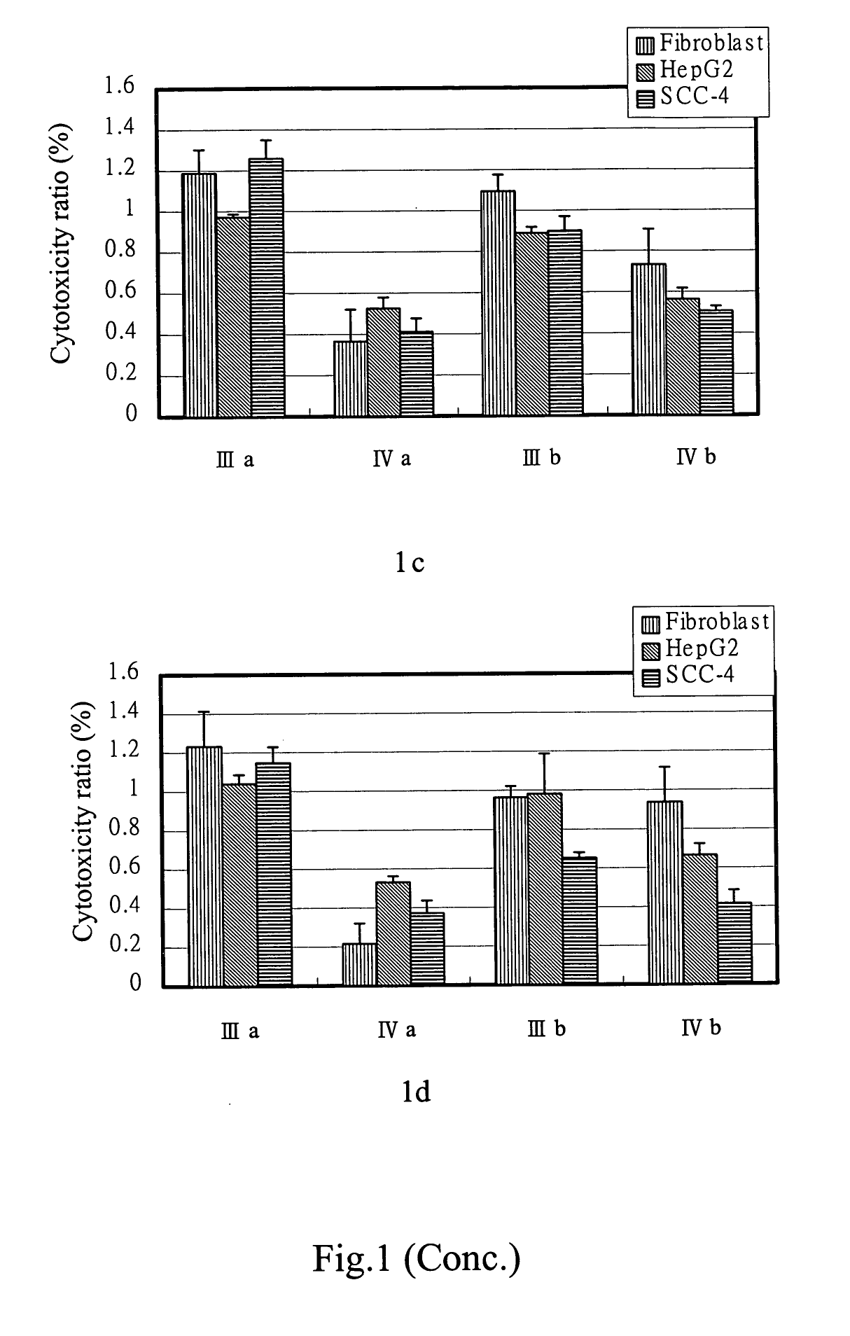

FIG. 1 was the result of the effect of phototoxicity on human foreskin fibroblast cells, HepG2/C3A and SCC-4 cancer cells after treatment of 1.25 μg/ml of 8 compounds for 150 minutes, and irradiation for 20 minutes (FIGS. 1a, 1c) or 30 minutes (FIGS. 1b, 1d) by MTT assay.

According to FIG. 1a and FIG. 1b, compound □a was toxic to liver carcinoma cells; the activity decreased 30% after irradiation for 20-30 minutes.

There is no significant toxicity to the 3 treated cells with compound Ia and Ib after irradiation for 20 or 30 minutes. Compound □b showed toxicity to oral cancer cells since the cell activity decreased 30% after irradiation for 20 minutes. Based on FIGS. 1c and 1d, compound III a and IV b were more toxic to liver cancer cells—HepG2/C3A since the activity decreased 20% after irradiation for 20-30 minutes. Compound III b and IV b also showed toxicity with the decreasd activity of oral cancer cell line—SCC-4 20-50% after irradiation for 20-30 minutes. Regardless of irradidation times, the activities of 3 cell lines treated with compound IV a showed no decrease. In addition, compound III a showed no significant toxicity to oral cancer cells either.

### EXAMPLE 5

Cell survival rate can be analyzed with Methylene blue staining. Ten line—HepG2/C3A, and oral carcinoma cell line—SCC-4 were placed into a 6-well cell culture plate. The human foreskin fibroblast cells were cultured in MEM medium (Gibco 61100-061) with 10% FBS. The liver cancer cell line—HepG2/C3A were cultured in DMEM medium (Gibco 12100-046) with 10% FBS. The oral cancer cell line—SCC-4 were cultured in DMEM/F12 medium (Gibco 12100-024) with 10% FBS. All cells were cultured overnight at 37° C., 5% Co₂ allowing cell attachment.

The cultured cells were rinsed with PBS twice, and serum free medium were then added into each well for cell culturing. The cells were treated with 1.25 μg/ml of 8 compounds for 150 minutes in the dark. After treatment, the drug solution in each well was washed off and the cells were rinsed with PBS twice, the PBS was replaced with serum free medium. The cells were then irradiated with 680 nm red-light for 20 minutes (accumulated energy 16 J/cm²) or 30 minutes (accumulated energy 24 J/cm²).

After irradiation, the serum free medium was replaced with medium containing 10% FBS and then continued culturing. The Methylene blue staining assay was performed 2 days after irradiation. 0.1 ml of 0.5% methylene blue (dissolved in 50% v/v ethanol/water (Sigma) before use) was applied to the cultured cells. 30 minutes later, the blue color of the supernatant was washed off with distilled water until the supernatant was clear, and 0.1 ml of 0.5 % SDS solution (sodium dodecyl sulfate, SDS, Sigma) was added to dissolve the methylene blue inside the cells. The ELISA reader (SOFTmax PRO) after 1 hour. All experiments were performed in triplicate, and the mean values were calculated. Cell survival ratio=absorbance_{(compound treated+light irradiation)}/absorbance_{(no compound treated+light irradiation)}.

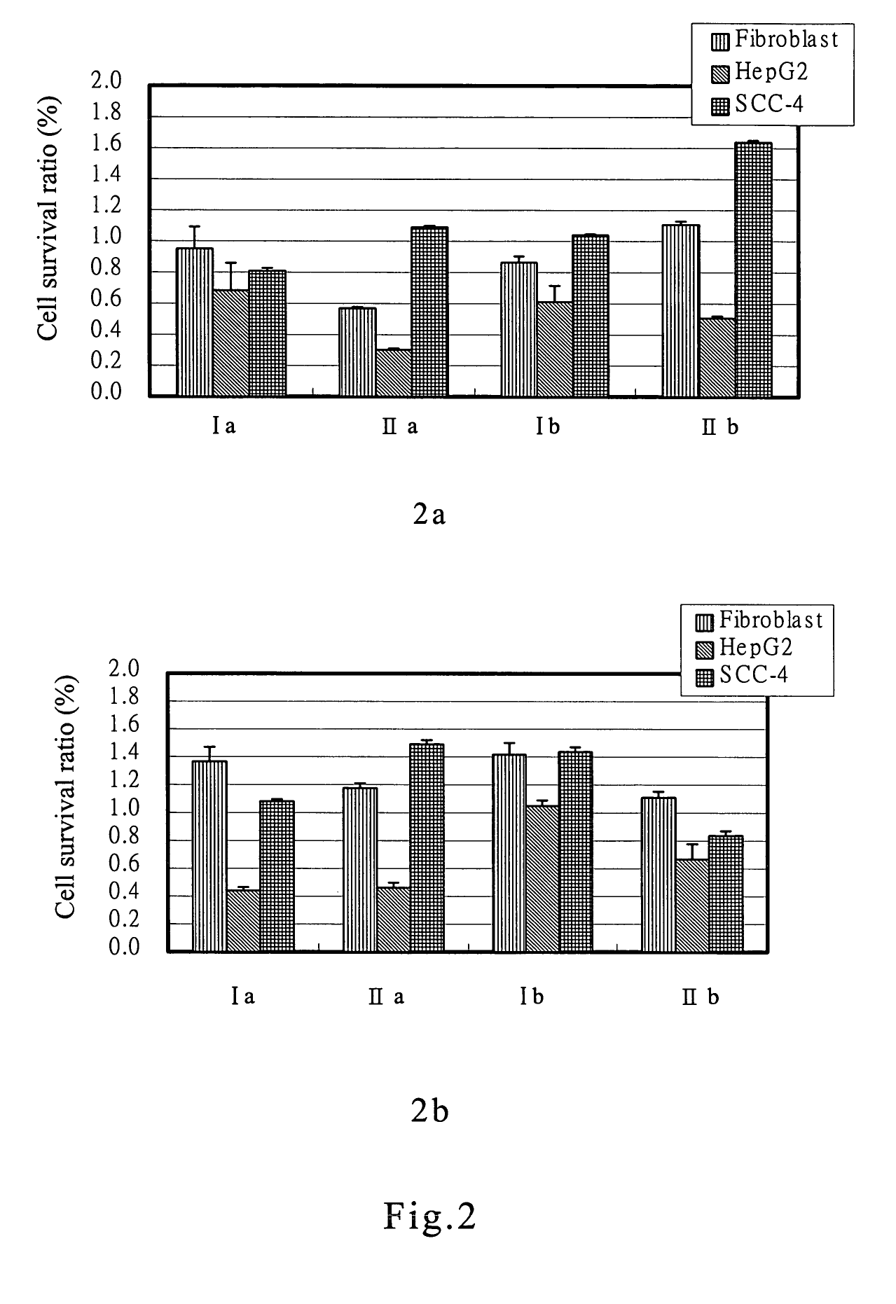

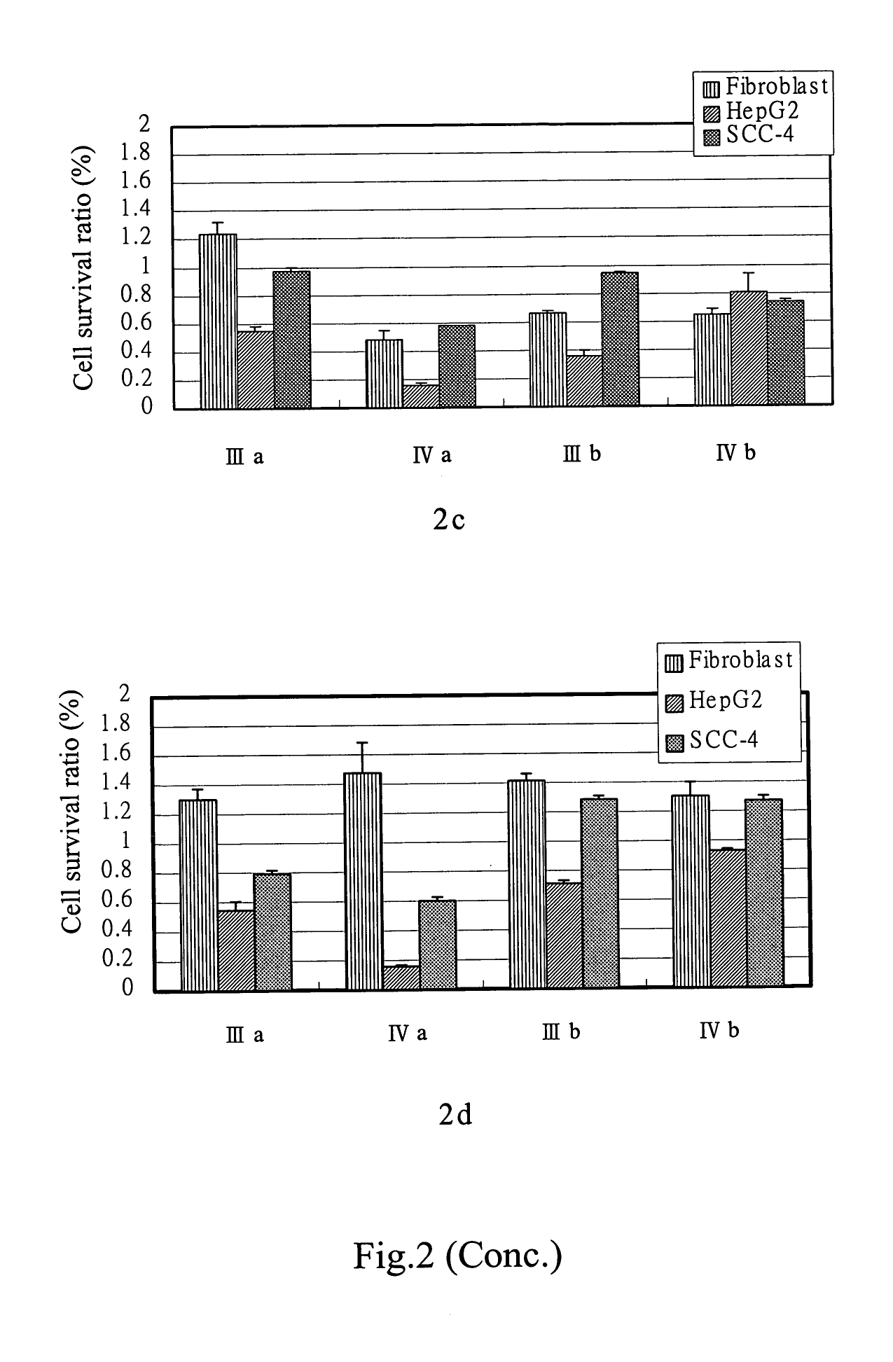

Methylene blue staining assay was performed on human foreskin fibroblast cells, HepG2/C3A and SCC-4 cancer cell lines after they were treated with 1.25 μg/ml of 8 compounds I a, I b, II a, III b, III a, III b, IVa and IVb for 150 minutes and irradiated for 20 minutes (FIGS. 2a, 2c) or 30 minutes (FIGS. 2b, 2d) to determine the survival ratio of cells.

Compounds with formulas I a, I b, II a, and II b showed a significant toxicity to liver hepatoma cells after the cells were irradiated for 20-30 minutes. According to FIG. 2b, compounds I a and II b showed phototoxicity to oral carcinoma cell line—SCC-4. FIG. 2d showed that compounds III a, III b, IVa and IVb had phototoxicity to both liver hepatoma cells and oral carcinoa cells after irradiation for 30 minutes. According to FIGS. 2c and 2d, the data indicated that 4 compounds of III a, III b, IVa and IV b had more phototoxicity to liver hepatoma cells than that of oral carcinoma cells.

### EXAMPLE 6

Fluorescent microscopy was used to examine the intactness of nucleus and mitochondria of a drug treated cell. The coverglasses were soaked in 70% alcohol overnight and were sterilized by passing through the plate, and 4×10⁵ cells (e.g. human foreskin fibroblast, liver hepatoma cell line—HepG2/C3A, and oral carcinoma cell line—SCC-4) were seeded into each well. The cells were incubated overnight in an environment of 37° C., 5% CO₂ for cell attachment.

The cultured cells were washed with PBS twice, serum free medium was added into each well. The cells were treated with 1.25 μg/ml of 8 different compounds for 120 minutes in the dark. After treatment, the drug solution was washed off and then the cells were rinsed twice with PBS. The cultured medium was replaced with serum free medium. The cells were subjected to irradiation with 635 nm red-light for 20 minutes (accumulated energy 16 J/cm²) or 30 minutes (accumulated energy 24 J/cm²). 0-24 hours after irradiation, the cells were then fixed with 2 ml of 2% formaldehyde, and the nuclei were stained for 5 minutes with 10 μl DAPI (4′,6′-diamino-2-phynyindole, Sigma). Rhodamine 123 (2-(6-amino-3-3H-imino-3H -xanthen-9-yl) benzoic acid methyl ester, Sigma) was used to stain mitochondria. DAPI or Rhodamine 123, and formaldehyde were washed off with PBS. The coverglass attached with cells was preserved in PBS.

DAPI stained cells were observed byfluorescent microscope (Leica) (excited with ultraviolet light), and the image of nuclei would be blue. The cells stained with Rhodamine 123 were observed with green exciting light, and the image of mitochondria would be red.

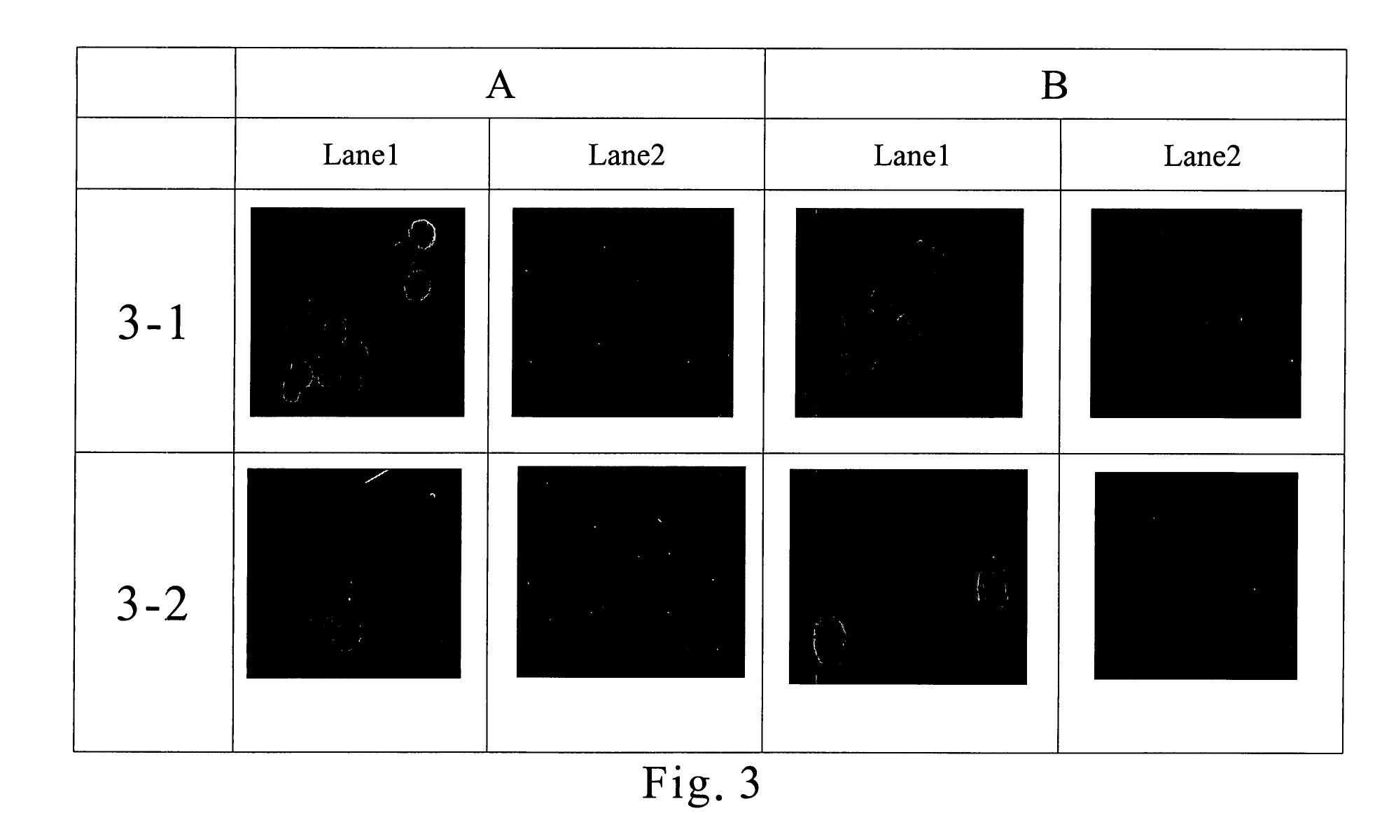

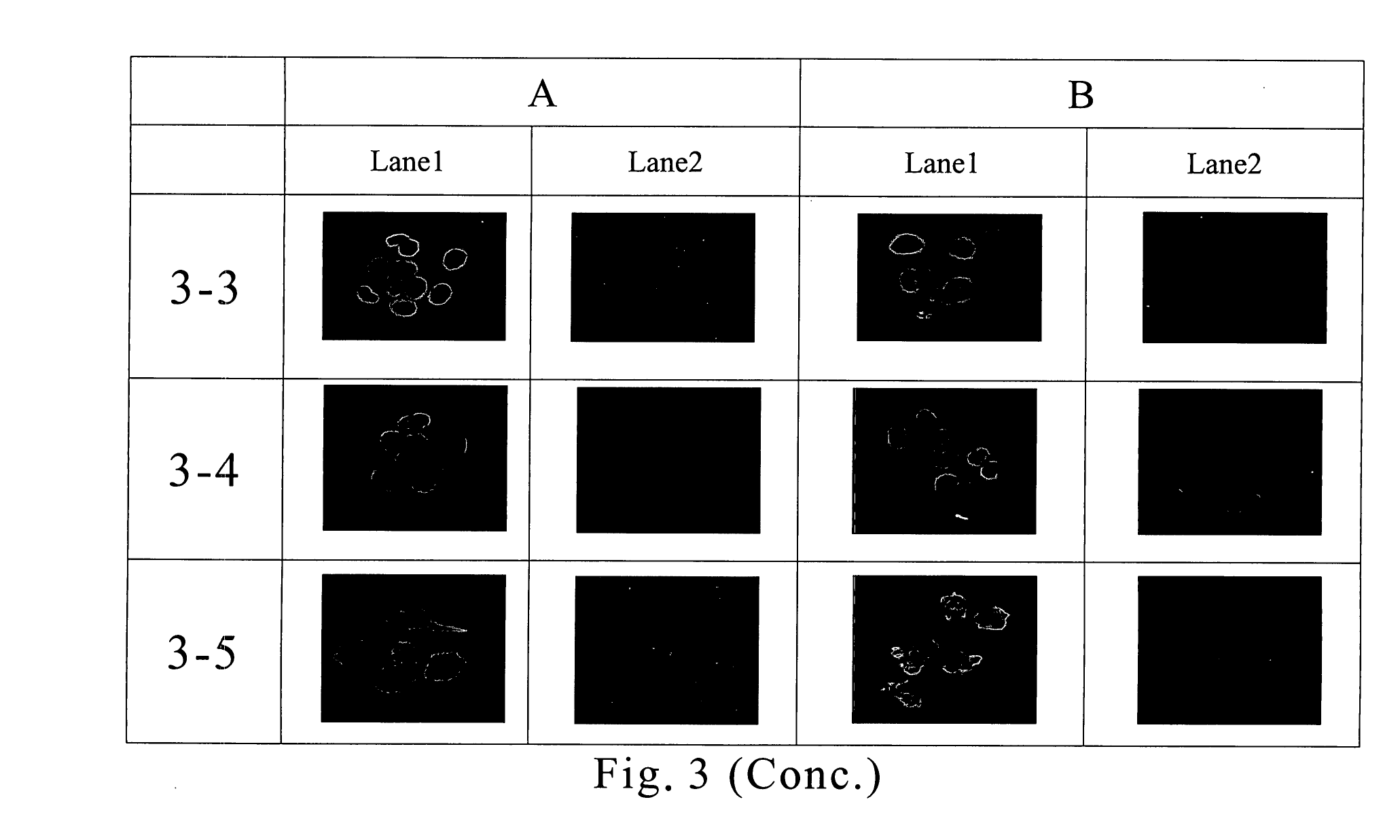

According to FIG. 3, panel A represents samples treated with compounds and irradiation. Lane 1 shows the results of DAPI staining, lane 2 shows the results of Rhodamine 123 staining. Row 3-1 indicates the morphology of liver hepatoma cells treated with compound □a, and the post-irradiation time was 8 hours. Row 3-2 indicates the morphology of oral carcinoma cells treated with compound □a, and the post-irradiation time was 8 hours. Row 3-3 indicates the morphology of liver hepatoma cells treated with compound IV a, and the post-irradiation time was 4 hours. Row 3-4 indicates the morphology of liver hepatoma cells treated with compound IV b, and the post-irradiation time was 24 hours. Row 3-5 indicates the morphology of oral carcinoma cells treated with compound IV b, and the post-irradiation time was 8 hours. According to FIG. 3, destruction of the nuclei and mitochondria were observed from 4 to 8 hours after the cells were treated with compounds. The cell morphology was gradually destroyed, and the integrity of the nucleus was lost. Eventually, Rhodamine 123, the dye used to stained mitochondria, was no longer localized inside the organelles and leaked out from the cells, therefore, the whole medium showed a red color background.

Although the present invention has been explained in relation to its preferred embodiment, it is to be understood that many other possible modifications and variations can be made without departing from the spirit and scope of the invention as hereinafter claimed.

1. A method for treating liver cancer through photodynamic therapy, comprising administrating a subject in need thereof an effective amount of formula I: 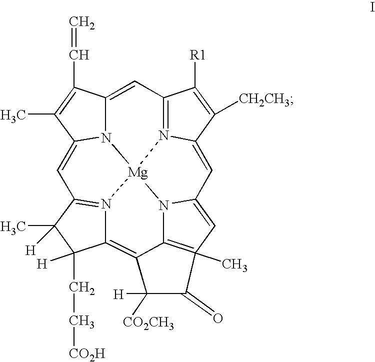
wherein, R1 is an alkyl or an aldehyde with carbon atoms no more than 2; said cancer is liver cancer.

2. The method according to claim 1, wherein R1 is CH₃.

3. The method according to claim 1, wherein R1 is CHO.

4. The method according to claim 1, wherein the spectral regions effectively for said photodynamic therapy is in a range of 600-800 nm.

5. A method for treating cancer through photodynamic therapy, comprising administrating a subject in need thereof an effective amount of formula ε, formula III and formula IV: 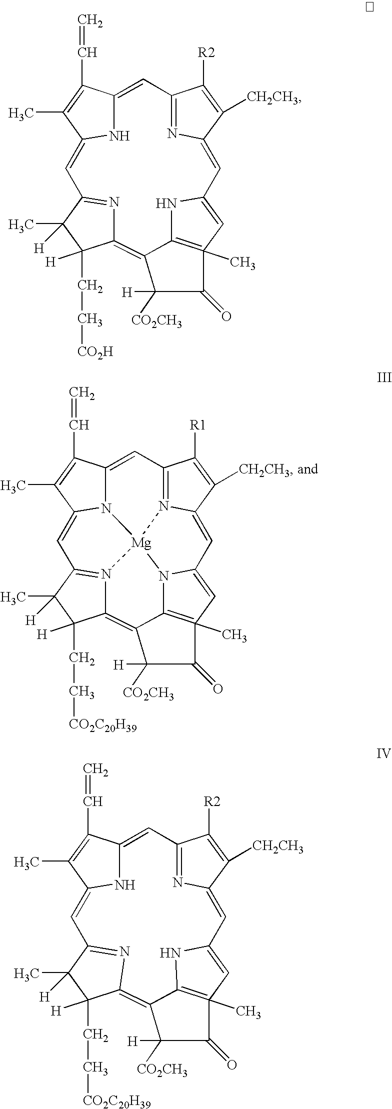
wherein, R1 and R2 is independently an alkyl or an aldehyde with carbons no more 2; said cancer is oral cancer or liver cancer.

6. The method according to claim 5, wherein R1 is CH₃.

7. The method according to claim 5, wherein R1 is CHO.

8. The method according to claim 5, wherein said cancer is oral cancer.

9. The method according to claim 5, wherein said cancer is liver cancer.

10. The method according to claim 5 wherein the spectral regions effectively for said compound is in a range of 600-800 nm.

11. The method according to claim 5, wherein said photodynamic therapy is for use in diagnostic and site location of cancer cells.

12. A pharmaceutical composition for cancer photodynamic therapy comprises at least one compound of formula I, formula □, formula III and formula IV: 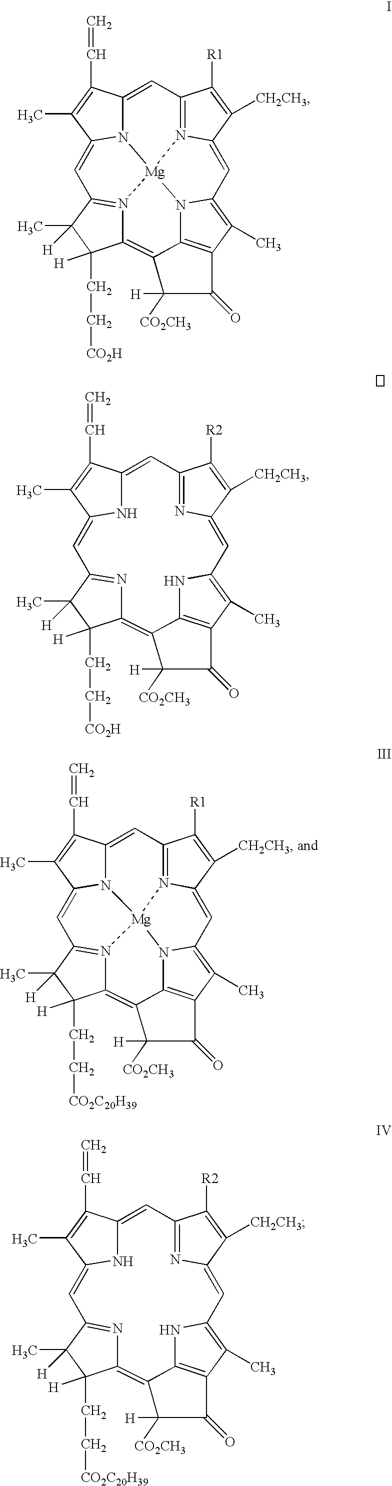
wherein, R1 and R2 is independently an alkyl or an aldehyde with carbon atoms no more than 2; said cancer is oral cancer or liver cancer.

13. The composition according to claim 12, wherein R1 is CH₃.

14. The composition according to claim 12, wherein R1 is CHO.

15. The composition according to claim 12, wherein the spectral regions effectively for said compound is in a range of 600-800 nm.
